(19) United States
(12) Patent Application Publication
(10) Pub. No.: US 2005/0240162 A1
(43) Pub. Date: Oct. 27, 2005

(54) Eye treatment device

(76) Inventors: Wen-Pin Chen, Taipei (TW); Naiying Jean, Taipei (TW)

Correspondence Address: Kai Chih Industrial Co., Ltd., P. O. Box No. 6-57, Junghe, Taipei, 235, TW

(21) Appl. No.: 10/828,196

(22) Filed: Apr. 21, 2004

(51) Int. Cl. A61M 35/00

(52) U.S. Cl. 604/298

(57) ABSTRACT

An eye treatment device that atomizes a medicament to form a liquid mist, which can then be directly sprayed onto the eyes of a user, and embraces a primary configuration including a cover member configured on a main body and adapted to cover periphery of the eyes, and a liquid mist exciter device configured to be at closest distance from the cover member. The exciter device utilizes a piezoelectric ceramic oscillator to actuate percussion boards, and liquid mist output sides of the percussion boards face the cover member, thereby forming a configuration that realizes closest separation from the eyes of the user and a high-speed cooling effect. The eye treatment device thus enables direct spraying of the liquid mist onto the eyes.

### BACKGROUND OF THE INVENTION

(a) Field of the Invention

The present invention relates to an eye treatment device, and more particularly to the treatment device that provides medicinal or physio-treatment of the eyes therewith. The present invention primarily utilizes a piezoelectric ceramic oscillator to actuate percussion boards with high frequency oscillations, whereupon the percussion boards excite a mechanical oscillation percussion on channeled liquid film to form atomized liquid mist micro-droplets, and thereat the atomized liquid mist micro-droplets are sprayed from a point closest away from the eyes of a user. The eye treatment device of the present invention further provides a configuration that retains structural composition of medicament employed, and realizes low power consumption and high efficacy, and thereby economizes on power usage while expediting an effective operating procedure.

(b) Description of the Prior Art

After contact lens were publicly introduced, though benefiting a great many wearers of spectacles, however, many successive problems developed from wearing such contact lens, because upon wearing the contact lens, moisture content in the eyes of the wearer continually evaporates, and thus surface of the contact lens becomes dry as a result, and the wearer of the contact lens feels uncomfortable, and experiences hazy eyesight, and is especially serious when the wearer of the contact lens is located in an environment which is unclean and dry or the wearer of the contact lens suffers from tears hyposecretion.

Furthermore, procedure for cleaning upkeep and sterilization of the contact lens is also demanding and complicated. As for routine purchases that can bring a glistening to eyes of the wearer of the contact lens, including eyewash or medicinal tablets for taking care of the contact lens, such can be briefly summarized as follows:
1. Cleaning liquid, which provides good cleaning effectiveness, and realizes a beneficial sterilization effect, and which can eliminate microorganisms, including amoeba strains.
2. Physiological saline, which is employed to prevent bacteria from breeding.
3. De-albumen tablets: classified as usage for enzyme cleaning, and which decompose and eradicate albumen accumulated on the surface of the contact lens.
4. Hydrogen peroxide, which is employed to provide the sterilizing effect. However, effectiveness of the hydrogen peroxide attenuates upon coming in contact with air and high temperature.

In light of the aforementioned medicaments required for the upkeep of the contact lens, though some are effective and are provided with particular functionality, however, a singular characteristic common to each method of administering the medicaments is namely a necessity to remove the contact lens in order to administer soaking of the contact lens, and more particularly is a requirement for a customized device to deploy rinsing of the contact lens, wherewith compete effectiveness of the medicaments can only then be achieved.

Prior publicly announced Taiwan patents, for instance No. 83204854 “Face Mask Providing Medicament Atomization Treatment for Eyes and Nose), and No. 91207965th (Steam Massage Device for Eyes), wherein, the “Steam Massage Device for Eyes” utilizes a steam generator and a heater, wherewith liquid is transformed into steam, which is thereupon sprayed out from a nozzle. Wherein, if medicaments are employed in the steam generator, because of a heat reaction, structural composition of the medicaments is damaged, and thus disadvantageous for usage in medicinal treatment of the eyes.

Whereas the face mask as disclosed in patent No. 83204854 is basically configured in a shape of the eyes and nose portion of a user, and thereby tight fitting when worn. Outward discharge is provided by means of a tee device having a switch and one entry passage. The entry passage connects with an atomization sprayer through a conduit, thereby necessitating atomized spray to pass through the connecting conduit and thereon to the entry passage, whereupon a selection is made with the switch of the tee device to direct the atomized spray to the eyes or nose of the user. Wherein, because the connecting conduit is of specific length, a mist formed by the atomization sprayer passing through the connecting conduit therefore produces a liquid damping effect on a wall of the conduit. Moreover, because of the damping effect, the mist condenses and forms a liquid that disadvantages pressure spraying of the mist. Furthermore, if the medicament employed is changed, remnants of eyewash employed in prior usage will remain, and is difficult to clean away.

Furthermore, the face mask covers both the eyes and nose portion of the user, thus If the user only desires to attend to the eyes portion, unnecessary burden is placed on the nose, and breathing will be even more difficult as a result. Moreover, gas exhaled by the user will directly pollute the face mask. In addition, the face mask completely covers a top half of the face. If attending only to the eyes, then the face mask is oversized, and quantity of medicament required is correspondingly increased, thus resulting in wastage of the medicament.

### SUMMARY OF THE INVENTION

The present invention provides for spraying atomized mist micro-droplets from a mist generator to the eyes of a user through shortest distance therebetween, while ensuring fineness of the atomized mist micro-droplets arriving at the eyes is retained. An embodiment of the present invention further comprises an ultrasound atomizer utilizing low power, and which atomizes eyewash or liquid to form the liquid mist micro-droplets of 2-10 microns in size. An ultraviolet light-emitting diode irradiates ultraviolet rays that realize production of traces of ozone O₃, thereby attaining effectiveness of sterilization therewith. After the aforesaid processing, the liquid micro-droplets are sprayed directly onto contact lens to implement cleaning, lubrication and sterilization thereof. Because a preferred embodiment of the present invention only requires an extremely low power consumption of approximately 0.5 w, therefore a battery can be used as a power supply. Furthermore, volume size of the present invention is minimized to dimensions of conventional goggles, and therefore facilitates portability, and is thus suitable to implement cleaning and treatment at anytime and anywhere, thereby furnishing the present invention with extreme convenience of usage.

The present invention primarily comprises the optimized ultrasound atomizer of low-power requirements, wherewith eyewash or liquid can be transformed into micro-droplets of 2-10 microns in size. The ultraviolet light-emitting diode (UV LED) is then utilized to irradiate the micro-droplets, thereupon simultaneously generating traces of ozone O₃, and because of extreme small size of the micro-droplets, maximum surface area of the micro-droplets is irradiated, and thus reaction time is exceedingly short, moreover, only extremely small traces of ozone O₃ of approximately 0.01-0.015 ppm are required. After the micro-droplets have undergone processing by the ultraviolet rays and the ozone O₃ the micro-droplets are atomized and uniformly sprayed onto the contact lens. Furthermore, powerful oxidization capabilities of the ozone O₃ is utilized to achieve sterilization, which thereby eradicates microorganisms and decomposes albumen, thereby eliminating build-up of the albumen. Because the atomized micro-droplets are utilized to achieve high-speed impact on a surface, effective cleaning away of dirt is thereby realized, and relieves the user from having to add droplets of the eyewash to the eyes, which because of blinking of the eyes to light reflection while adding the eyewash, effectiveness of the eyewash is affected.

Accordingly, the present invention is configured to comprise a cover member adapted with specifications to cover periphery of the eyes, and the cover member is positioned at closest distance from a liquid mist exciter device, thereby a straight line is formed from the liquid mist exciter device to the eyes of the user via through holes defined in the cover member. The liquid mist exciter device utilizes a piezoelectric ceramic oscillator providing high-frequency oscillation to directly actuate percussion boards, and thereby enables the percussion boards to spray the liquid mist from an output side directly towards the eyes of the user. A liquid input side faces liquid films formed by liquid guide members, which thereat allows the liquid films to undergo micro-excitation. The liquid guide members channel liquid from a liquid chamber, and after exploiting a cooling effect the liquid mist is directly sprayed towards the eyes via the closest distance, thereby realizing treatment of the eyes. The aforementioned disclosures are thus a primary objective of the present invention.

Another objective of the present invention is to further provide a reaction chamber having a sterilization effect, and configured in a partition between the liquid mist output side of the percussion board and the through hole of the cover member. The ultraviolet light-emitting diode is configured within the reaction chamber, and which is utilized to generate the ultraviolet rays therefrom. Because the liquid mist produced is of a micro-droplet form, the micro-droplets present a maximum reaction area to the ultraviolet rays, and thus maximize oxidization and effective sterilization.

A third objective of the present invention is to utilize an additional inpouring method to accommodate requirements for inpouring of the liquid, and facilitating quick replacement of the medicament therewith. Furthermore, a container is employed, and a piezoelectric oscillator is configured in a base of the container so as to upwardly face a mouth of the container, and whereby the liquid mist is directly channeled to the cover member configured atop the liquid container, whereupon the liquid mist is sprayed through the through holes of the cover member.

A fourth objective of the present invention is to further provide a configuration that embodies two liquid channels diverging from the liquid chamber, which respectively channel the liquid to the two percussion boards, whereupon the two percussion boards actualize synchronous atomization of the liquid mist, and thereby facilitates synchronous treatment of the two eyes of the user.

A fifth objective of the present invention is to further provide configuration of two percussion boards of equal mass on two opposite side wings of the oscillator, thereby enabling the single central oscillator to actuate synchronous oscillation of the two percussion boards, and thus realize a configuration which provides synchronous actuation of two percussion boards from the single oscillator.

A sixth objective of the present invention is to further configure an insert and extract assemblage between the percussion boards and the oscillator, which therewith facilitates replacement or cleaning of the percussion boards. Wherein requirements may demand replacement of the percussion boards with percussion boards having grooves of varied form defined in surfaces of the percussion boards, and wherewith modifies output quantity of the liquid mist or choice of micro-droplets formed.

A seventh objective of the present invention is to further utilize connecting rings to assemble and secure the liquid guide members to the liquid chamber. The connecting rings thereby enable convenient replacement or cleaning of the liquid guide members.

An eighth objective of the present invention is to further configure a clip fastening mechanism to fasten the liquid chamber to the main body, and thereby facilitate replenishing the liquid in the liquid chamber or replacing with different medicinal eyewash.

A ninth objective of the present invention is to provide an embodiment wherein apart from percussion orifices defined in the percussion boards, line-shaped percussion grooves can be further defined in the surfaces of the percussion boards, whereby width of the percussion grooves can be defined so as to be smaller than width of possible dust particles, and length of the percussion grooves are defined so as to be far longer than length of the dust particles, thereby preventing blockage by the dust particles. Furthermore, a distribution of the percussion grooves having varied shapes or alternate arrangement of shaped lines including curves, straight lines, angled lines, and so on, are utilized to realize spraying of varied atomized liquid mist micro-droplets.

An tenth objective of the present invention is to configure a PS2 or USB connector on the main body, and which provides for externally connecting to a host computer that can supply corresponding electric power, thus when operating the computer, such a connection facilitates simultaneous acquisition of the liquid vapor that therewith realizes health care of the eyes.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a general view of an embodiment according to the present invention.

FIG. 2 shows a cross sectional structural view according to the present invention.

FIG. 3 shows a simplified cross sectional view of an embodiment utilizing a disc-shaped oscillator according to the present invention.

FIG. 4 shows a working schematic view of a reaction chamber according to the present invention.

FIG. 5 shows an elevational view of another embodiment with provision for synchronous treatment of two eyes according to the present invention.

FIG. 6 shows an elevational view of a configurational relationship of an exciter device synchronously actuating two percussion boards and a liquid chamber according to the present invention.

FIG. 7 shows a cross sectional schematic view of an assemblage of a connecting ring according to the present invention.

FIG. 8 and FIG. 8A shows a cross sectional schematic view and an partial enlarged view of the percussion boards clipped to an oscillator according to the present invention.

FIG. 9 shows a top schematic view of the percussion boards further defined with percussion grooves according to the present invention.

### DETAILED DESCRIPTION OF THE PREFERRED EMBODIMENTS

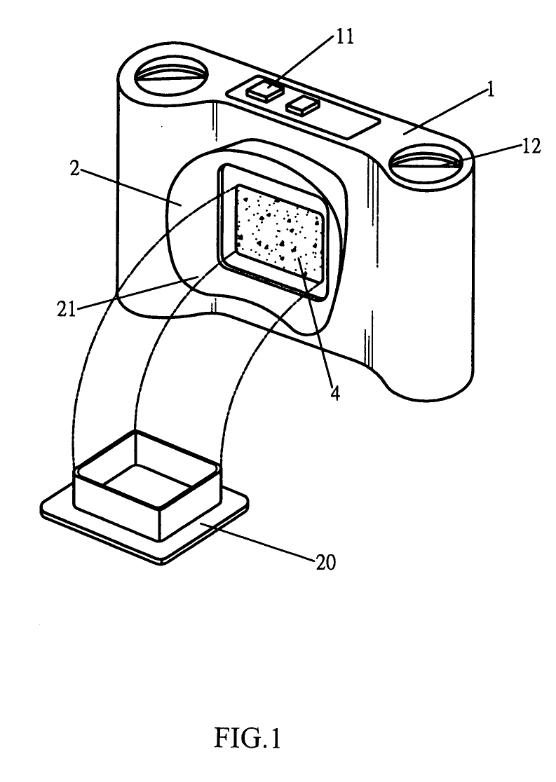

Referring to FIG. 1, which shows an embodiment of the present invention primarily comprising a box-like form main body 1. A cover member 2 is configured on a side of the main body 1, and consists of a flexible rim that can be lightly pressed over surrounding area of an eye of a user. A through hole 21 is defined within the cover member 2, and a liquid mist exciter device 4 is directly configured within the through hole 21. Upon storing away the present invention, an anti-dust cover 20 is placed over the through hole 21, thereby covering and preventing dust from entering the through hole 21. Compartments 12 are configured on sides of the main body 1, and a control switch 11 is configured atop the main body 1. The compartments 12 provide for fitting of batteries therein or can be suitably adapted for disposing of reciprocal liquid storage holders therein. The control switch 11 controls functioning of the exciter device 4. The present invention as disclosed embodies a design of exquisite configuration and ingenuity.

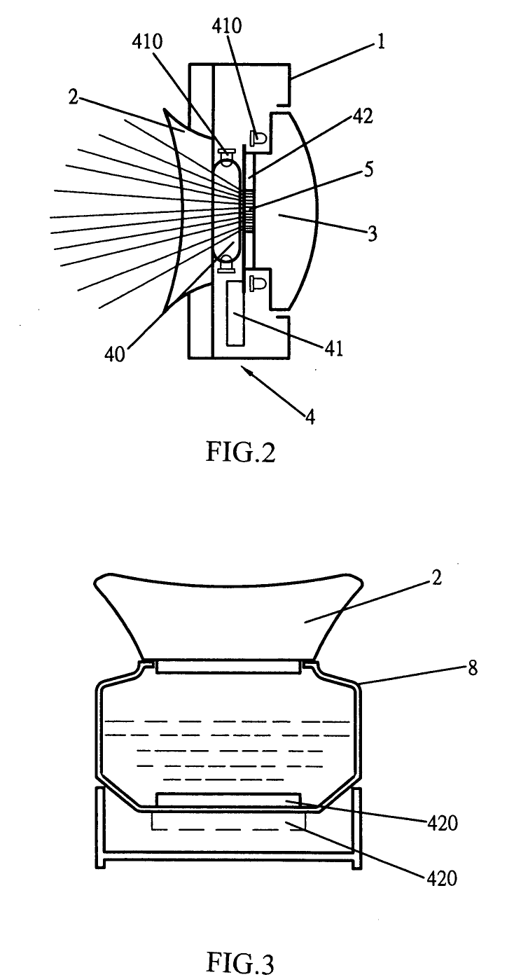

The present invention utilizes a piezoelectric ceramic oscillator 41 (see FIG. 2) configured within the main body 1, and which directly actuates a liquid mist percussion board 42. A liquid mist output side of the percussion board 42 is configured so as to face and be at shortest distance from the cover member 2. Furthermore, a liquid guide member 5 is configured on a liquid input side of the percussion board 42, and which is deployed to channel the liquid within a liquid chamber 3 towards the percussion board 42 by means of capillarity. In addition, mutual interfacial cohesion between the liquid guide member 5 and the percussion board 42 enables formation of a liquid film, and provides for the percussion board 42 to effectively realize atomization spraying of the liquid film. Operating distance for liquid atomization spraying according to the present invention is approximately 8-10 cms (tested indoors in absence of draughts). Therefore, according to configuration of the present invention as disclosed, the liquid mist exciter device 4 is configured at the shortest distance from the cover member 2, and atomized liquid is sprayed towards the cover member 2. Moreover, because the liquid is channeled directly through the shortest distance from the liquid chamber 3 to the cover member 2 by means of the liquid guide member 5, thus a viscous effect of the liquid is prevented from occurring during course of the liquid mist being sprayed, and thereby ensures eventual outspraying liquid mist micro-droplets will not condense, as well as ensuring uniform fineness of the mist micro-droplets.

Force from the atomized spray is sufficient to enable the liquid mist to produce a striking impact mechanical energy, which can be equally controlled and modulated by means of a switch. The exciter device 4 actuates the percussion board 42 by means of the piezoelectric ceramic oscillator 41 providing 200-300 KHZ high frequency oscillations. Kinetic energy of the high frequency oscillations thereby enables direct atomization of the liquid. During course of atomization, because of high speed of the high frequency oscillations, heat energy generated is quickly carried away by rapid exchange of the liquid being atomized, and corresponding cooling results thereof. Hence, temperature of the liquid mist produced is slightly lower than temperature of the liquid in the liquid chamber 3. In principle, the exciter device 4 can be deemed to provide a cooling function, and thus will not alter medical properties of the liquid within the liquid chamber 3.

The present invention further comprises a sterilizing reaction chamber 40 configured in a partition between the percussion board 42 and the cover member 2. The sterilizing reaction chamber 40 is basically an inner circular body, and a sterilization function of the reaction chamber 40 is based on an ultraviolet sterilization effect. Ultraviolet light-emitting diode lamps 410 are utilized to generate ultraviolet rays, wherewith the ultraviolet rays irradiate the liquid mist being emitted through the reaction chamber 40. Along with increasing progress in semiconductor fabrication, there has also been substantial progress in development of light-emitting diodes, thus, taking the initiative, the present invention utilizes the ultraviolet light-emitting diode lamps 410 to produce ozone O₃ within the reaction chamber 40, and because of high-efficiency of the ultraviolet light-emitting diode, wavelength uniformity is high and volume size of the ultraviolet light-emitting diode is small, therefore the ultraviolet light-emitting diode lamps are utilized as ultraviolet light sources, which thereby realizes a portable product that provides saving in electrical power and ingenuity in design. In addition, the ultraviolet light-emitting diode lamps 410 operate in coordination with spheroidal design of the reaction chamber 40 to prevent the ultraviolet rays from scattering and leaking out. The ozone O₃ produced within the reaction chamber 40 is atomized and sprayed outwards. A 50% alternating light and extinguish DUTY CYCLE is utilized to power the light-emitting diode lamps 410, thereby minimizing decrease in light-emitting efficiency of the light-emitting diode lamps 410 affected by rise in temperature of same. Furthermore, because diameter of the liquid micro-droplets is only 2-10 um, therefore a unit cubic measurement of the liquid after being atomized to form liquid mist micro-droplets, because the liquid has undergone decomposition of form and structure to produce the micro-droplets, total surface area the atomized liquid mist comes in contact with is extremely large. Hence, only approximately 10 ppm of ozone O₃ is expended to achieve an effective sterilization reaction that is rapid and uniform. Similarly, the ultraviolet light-emitting diode lamps 410 can also be configurationally designed and deployed to cast ultraviolet rays towards transparent body of the liquid chamber 3, thereby enabling the liquid within the liquid chamber 3 to similarly receive the corresponding ultraviolet ray effect from the ultraviolet light-emitting diode lamps 410.

If the liquid chamber 3 is adapted with heat-resistant functionality, then hot liquid of high temperature can be stored therein. Upon the percussion board 42 being actuated, because of high velocity, the liquid mist produced from the high-temperature hot liquid is of correspondingly high temperature, which can therefore be employed for fomenting the face of the user, for instance, physiotherapy procedures of steam fomenting. The liquid chamber 3 can be further configured with a heating device. However, because an objective of the present invention is to provide for convenience of portability, and consideration has also been given for degree of labor required in manufacturing, thus primary inspiration behind the present invention is to provide a selective design that benefits convenience for outdoor usage.

Because electric power requirements for accurate operation of the exciter device 4 utilized by the present invention is approximately 50 mA, thus a connector can be further configured at a power supply input terminal, and which provides for connecting to a PS2, USB or equivalent port of a computer, thereby when operating the computer, the host computer provides for facilitating a supply of electric power to the exciter device 4 as provided by the host computer terminal.

The piezoelectric ceramic oscillator 41 of the present invention utilizes a linear conversion method to achieve objective of actuating a piezoelectric strip, and therefore the present invention adapts efficiency of an electric circuit to attain an efficiency η>85%. During implementation, the present invention utilizes a direct current working voltage of 3 VDC, whereby main power consumption is 0.5 watt-hour, which thereby achieves a standard atomization measurement of 100 cc per hour.

Furthermore, the electric circuit utilizes a CONSTANT VOLTAGE (CV), and by monitoring and controlling load lightness and power consumption of the exciter device 4, a load regulation error measurement within 1% can thereby be achieved, moreover, a line regulation range of at least ±50% can be attained.

In addition, a frequency constant and an output power constant can be altered by varying resistivity, thereby optionally regulating atomization state, and thus further providing the present invention with extreme convenience.

In order to further facilitate replacing the liquid, the present invention adapts a container 8 (see FIG. 3), whereby a piezoelectric oscillator 420 is configured in a base either internal or external to the container 8. Wherein, if the piezoelectric oscillator 420 is configured in the base external to the container 8, material the container 8 is made from is required to be provided with flexibility, thereby effectively allowing bearing of an entire load and oscillating of supporting liquid therewith, while realizing atomization of the liquid. Apart from the piezoelectric oscillator 420 adapting specifications of the piezoelectric oscillator 41 of the aforementioned (as depicted in FIG. 2) configuration, the piezoelectric oscillator 420 can be adapted to be of disc-shape form having axial amplitude of oscillation (see FIG. 3), and be employed and configured on the base interior to the liquid container 8. After inpouring of the liquid into the liquid container 8, mechanical energy of the axial amplitude of oscillation is transmitted through the liquid to surface of the liquid, whereupon interference of oscillating waves on the surface of the liquid realizes excitation of the liquid and produces liquid mist therefrom. Such a configuration can be utilized in situations where immovable atomization is applicable, for instance, for usage in a home or indoors, and so on. The cover member 2 is perpendicularly configured at closest distance to a mouth of the liquid container 8. The user adopts a face down position, and positions the eyes over the cover member 2, thereby allowing spraying of the eyes.

The aforementioned disc-shaped piezoelectric oscillator 420 can also be configured in the base external to the liquid container 8, thereby realizing reduction in processing work required for electrical insulation, and whereby the material the container 8 is made from is required to be provided with flexibility. Hence, the axial amplitude of oscillation of the disc-shaped piezoelectric oscillator 420 is transmitted through the base and the liquid of the liquid container 8, and synchronously achieves uniform oscillation thereof.

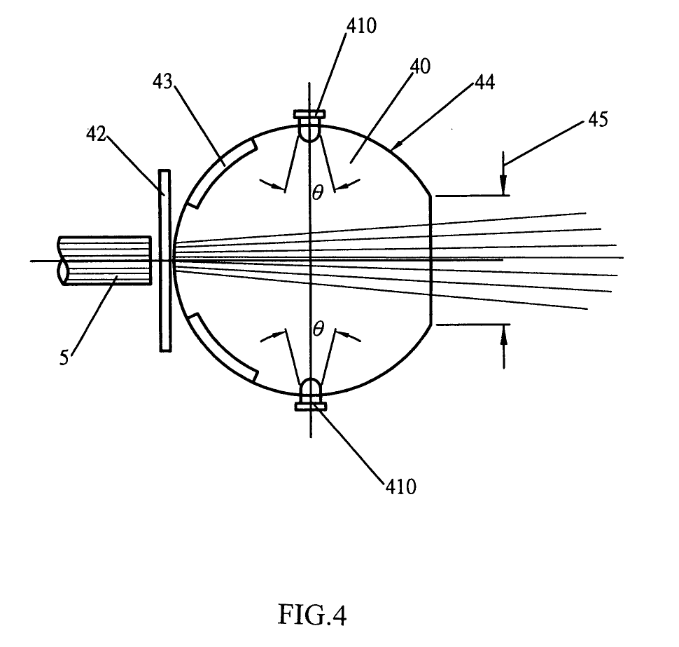

Referring to FIG. 4, which shows the reaction chamber 40 of the present invention configured basically in form of an inner body. A percussion board 42 is configured on a side of the reaction chamber 40. The liquid guide member 5 is configured on one side of the percussion board 42, and which is utilized as an input terminal-channel for the liquid to pass through. The ultraviolet light-emitting diode lamps 410 are oppositely configured within the reaction chamber 40, and which provide for emitting ultraviolet rays therefrom. When the liquid mist sprayed out by the percussion board 42 passes through the reaction chamber 40, thereat the ultraviolet rays irradiate maximum surface area of the liquid mist, which is then sprayed towards the face of the user through a contractile orifice 45. Furthermore, a shade panel is configured between the contractile orifice 45 and the ultraviolet light-emitting diode lamps 410, wherewith enables obstructing light source irradiation from the ultraviolet ray generators 410 from directly intruding the eyes. Because the reaction chamber 40 is configured as a circular body having specific capacity, the liquid mist flowing through allows for a partial anti-condensation effect, and the liquid that condenses can be wiped away, thereby cleaning away accumulation of the liquid. A liquid discharge device 43 can also be employed to channel out the remaining liquid. The liquid discharge device 43 can be so configured to exploit a capillary effect or any device configured to absorb liquid can be utilized, and is thus not described in further detail herein.

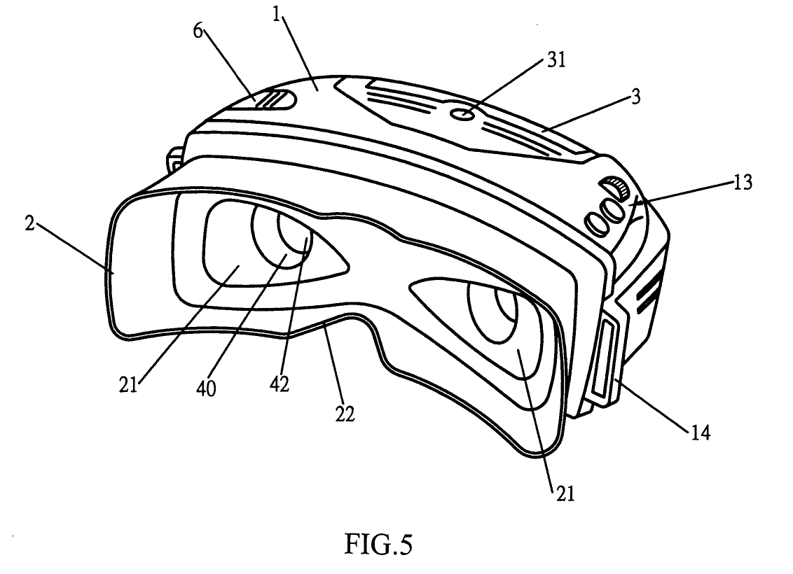

Referring to FIG. 5, which shows the main body 1 of the present invention adapted with a configurationally format defined with a pair of orifices, and comprising the lengthwise eye cover member 2 configured on the single main body 1, wherein the two through holes 21 are defined in the lengthwise eye cover member 2, and each of the through holes 21 provide for a passage with the percussion boards 42. The reaction chambers 40 can be respectively configured between the percussion board 42 and the through hole 21 in each side of the main body 1. A power supply device 6 is configured on one side of the main body 1, and a fastening method connecting the liquid chamber 3 is further configured to the main body 1 thereof. A pressure balance valve 31 is configured atop the liquid chamber 3 to respond to atmospheric pressure. A control switch 13 is positionally configured on one side of the main body 1, and which facilitates finger operation of the present invention therewith. Butt straps 14 are respectively configured on each of two sides of the main body 1, thereby enabling employment of any type of strap to securely fasten the present invention to the head of the user. Because power requirements of the present invention is extremely low, a battery providing 3 VDC is utilized or when being used at home, electrical voltage supplied at the home can be converted to direct current. In addition, any hand operated electric power dynamo can also be employed to provide electrical power requirements of the power supply device 6. Because the eye cover member 2 is of a form providing a lengthwise opening which is shaped to correspond with curve of the face of the user, hence, a recess 22 is centrally defined in a lower edge of the eye cover member 2 to correspond with bridge of the nose of the face of the user, thereby facilitating wearing, whereby the recess 22 provides an opening for positioning of the bridge of the nose therein. Furthermore, the eye cover member 2 is made from a material provided with flexibility, therefore lightly pressing a peripheral edge of the eye cover member 2 onto surface of the face of the user thereby realizes press-fitting of the eye cover member 2 onto the face of the user, and secure fitment thereof. The liquid chamber 3 can adapt a clip assemblage method to clip to the main body 1, whereby configuration of the clip assemblage utilizes a general mechanism as provided by a conventional coupling assemblage, and thus is not described in further detail herein.

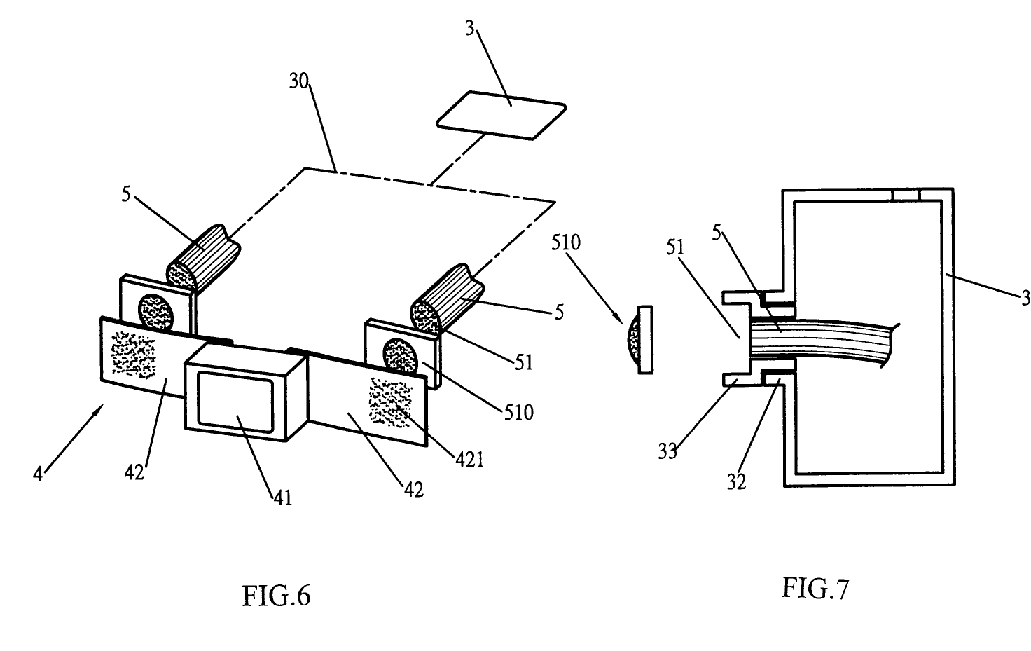

Referring to FIG. 6, which shows a configuration providing two liquid channels 30 diverging from the liquid chamber 3, and which separately provide for the liquid to be channeled to the two liquid guide members 5. After passing through the liquid guide members 5, the liquid thereupon forms the liquid film on each surface of end orifices 51 (see FIG. 7) by means of the capillary effect, and which thereby provide sources of liquid films for the percussion boards 42. The percussion boards 42 are correspondingly configured on opposite side wings of the piezoelectric ceramic oscillator 41, and so positioned so as to directly face the end orifices 51 of the liquid guide members 5. The piezoelectric ceramic oscillator 41 thereby synchronously actuates the two side percussion boards 42 which effectuate a percussion effect on the liquid passing through the liquid guide members 5. The liquid films formed after oscillation thereupon pass through percussion orifices 421 defined in the percussion boards 42, and thereat undergoes pressure slicing, which thus actualizes atomization of the liquid film. Structural configuration and mass of the two percussion boards 42 are equal, which thereby enables producing mist of uniform form. Accordingly, configuration of the exciter device 4 as disclosed above can be utilized for actualizing excitation within the reactor chamber as depicted in FIG. 4.

In addition, to order to accommodate different properties and characteristics so formed between the end orifices 51 and the percussion boards 42 by the liquid guide members 5, while still enabling the percussion boards 42 to attain actualization of liquid films of invariable form, a channeling diffusion piece 510 is respectively configured between the liquid guide member 5 and the percussion board 42 on each side of the exciter device 4. The diffusion pieces 510 also provide a liquid guide capillary function, whereby the diffusion pieces 510 utilized are of specific form, structure and material, and together with frontal interfaces of the percussion boards 42 can thereby provide the liquid film with specific form and structure, which can then furnish the percussion boards 42. Furthermore, reverse sides of the percussion boards 42 directly face the end orifices 51 of the liquid guide members 5. The liquid guide members 5 are fabricated from any material and configured such to channel and replenish supply of the liquid to the end orifices 51.

Referring to FIG. 7, which shows the liquid chamber 3 and the liquid guide members 5 assembled in an embedded type fashion, whereby the liquid guide members 5 are embedded into tube orifices 32 while outer extremities retain the end orifices 51. Connecting rings 33 are respectively configured between the tube orifice 32 and the end orifice 51 on each side of the exciter device 4, firstly to collar down the liquid guide members 5, and further realizing an assemblage on the tube orifices 32 of the liquid chamber 3, while also functioning to facilitate assembling and disassembling of the liquid guide members 5 from the liquid chamber 3, and thereby conveniencing insertion and extraction of the liquid guide member 5, as well as realizing waterproofing therewith. Accordingly, utilizing an insert and extract assemblage as disclosed facilitates replacing or cleaning of the liquid guide members 5, and employment of the connecting rings 33 also indirectly assist in waterproofing and maintaining airtightness between the liquid guide members 5 and the tube orifices 32.

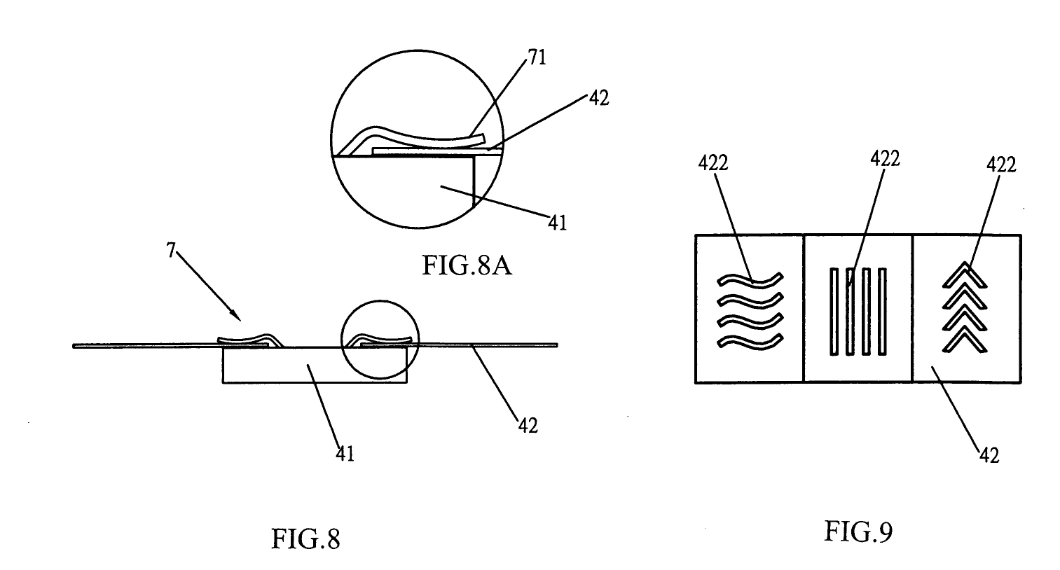

Referring to FIG. 8 and FIG. 8A, which shows an assemblage of the piezoelectric ceramic oscillator 41 and the percussion boards 42 according to the present invention, whereby soldering or any mode of adhesive bonding can be employed to join the piezoelectric ceramic oscillator 41 to the percussion boards 42. However, in order to further facilitate replacement of the percussion boards 42, the present invention can also employ a clip fastening mechanism 7 configured on a surface of the piezoelectric ceramic oscillator 41, which thereby provides a fastening method which conveniences replacement of the percussion boards 42. The clip fastening mechanism 7 utilizes clips 71 to clip down the percussion boards 42 thereof, and thereby an assemblage that accommodates convenient inserting and dismantling of the percussion boards 42 is actualized.

An objective of the insert and extract assemblage is to facilitate the user in dismantling and replacing or cleaning the percussion boards 42. If requirements demand, the percussion boards 42 can also be dismantled and replaced with percussion boards 42 defined with varied shaped-holes (see FIG. 9), whereby, apart from the percussion orifices 421 defined in the percussion boards 42, as aforementioned and depicted in FIG. 6, line-shaped percussion grooves 422 can be further defined in the surfaces of the percussion boards 42. The percussion grooves 422 can be shaped to form curves, straight lines, angled lines, and so on, wherein width of the percussion grooves 422 are defined such to be smaller than width of common dust particles (definition of dimensions and external form of the dust particles affects injury to the eyes), and length of the percussion grooves 422 are defined so as to be far longer than length of the dust particles. Hence, utilization of the line-shaped percussion grooves 422 realizes significant reduction in probability of the dust particles blocking the percussion boards 42. If a dust particle should block one of the percussion grooves 422, because the length of the percussion grooves 422 is far greater than the length of the dust particles, thus the dust particle will not affect compete obstruction, and unhindered atomization is ensured.

It is of course to be understood that the embodiments described herein is merely illustrative of the principles of the invention and that a wide variety of modifications thereto may be effected by persons skilled in the art without departing from the spirit and scope of the invention as set forth in the following claims.

1. An eye treatment device comprising a main body, a cover member configured to cover periphery of eyes, and through holes further defined in the cover member; a liquid mist exciter device is configured to be at closest distance from the cover member, and a piezoelectric ceramic oscillator is configured in the exciter device, and which is utilized to actuate percussion boards; liquid mist output sides of the percussion boards face the cover member, while liquid input sides are so configured to be close to end orifices of liquid guide members, and another end of the liquid guide members channel liquid from a liquid chamber; and therewith providing for spraying of eyes with liquid mist,

2. The eye treatment device according to claim 1, wherein reaction chambers are further configured in a partition between the percussion boards and the cover member, and ultraviolet light-emitting diode lamps are configured within the reaction chamber, and provide for generating ultraviolet rays.

3. The eye treatment device according to claim 1, wherein two liquid channels are configured to diverge from the liquid chamber, and which separately provide for the liquid to be channeled to the two percussion boards for further processing therewith.

4. The eye treatment device according to claim 1, wherein the two percussion boards are correspondingly configured on opposite side wings of the piezoelectric ceramic oscillator.

5. The eye treatment device according to claim 1, wherein a clip fastening mechanism utilizing clips configured between the piezoelectric ceramic oscillator and the percussion boards is adapted, which thereby provide a clip fastening method for assembling and disassembling the percussion boards.

6. The eye treatment device according to claim 1, wherein between the piezoelectric ceramic oscillator and the percussion boards, an adhesive agent or a metallic soldering method is utilized as a connecting configuration thereof.

7. The eye treatment device according to claim 1, wherein connecting rings are respectively further configured between the liquid guide members and the liquid chamber, and which are utilized to assemble and secure the liquid guide members to the liquid chamber.

8. The eye treatment device according to claim 1, wherein the liquid chamber can adapt a clip fastening mechanism to clip to the main body thereof.

9. The eye treatment device according to claim 1, wherein percussion orifices defined in surfaces of the percussion boards can be further defined as line-shaped grooves.

10. The eye treatment device according to claim 1, wherein a PS2 or USB connector is configured at a power supply input terminal, and which provides for connecting to a host computer.

11. An eye treatment device comprising a liquid container, in a base of which is configured the oscillator; the eye cover member adapted to cover the periphery of the eyes is directly configured atop a mouth of the liquid container, and the through holes defined in the cover member realize a passage through into the liquid container, and therewith providing an equipment that produces liquid mist which can be sprayed towards the eyes for treatment thereof.
